(19) United States
(12) Patent Application Publication
(10) Pub. No.: US 2024/0287597 A1
(43) Pub. Date: Aug. 29, 2024

(54) BIOMOLECULE ANALYSIS METHOD, BIOMOLECULE ANALYZING REAGENT, AND BIOMOLECULE ANALYSIS DEVICE

(71) Applicant: HITACHI HIGH-TECH CORPORATION, Tokyo (JP)

(72) Inventors: Rena AKAHORI, Tokyo (JP); Tatsuo NAKAGAWA, Tokyo (JP); Hai Huy NGUYENPHAM, Tokyo (JP)

(21) Appl. No.: 18/290,020

(22) PCT Filed: Jun. 15, 2021
(86) PCT No.: PCT/JP2021/022614

§ 371 Date: Nov. 9, 2023

(51) Int. Cl. C12Q 1/6869; G01N 27/04; G01N 33/487

(52) U.S. Cl. CPC C12Q 1/6869; G01N 27/04; G01N 33/48721

(57) ABSTRACT

A biomolecule analysis method of the present disclosure includes: preparing a biomolecule analysis device that includes a thin film having a nanopore with a diameter in a range of ±20% of a diameter of a biomolecule, a first liquid tank and a second liquid tank separated by the thin film, a first electrode disposed in the first liquid tank, a second electrode disposed in the second liquid tank, and a biopolymer degradation mechanism that degrades a biopolymer into the biomolecule; degrading a biopolymer into the biomolecule in the biopolymer degradation mechanism; and applying a voltage between the first electrode and the second electrode in a state where a measurement solution is enclosed in the first liquid tank and the second liquid tank, and measuring a current flowing between the first electrode and the second electrode, in which the measurement solution contains ammonium ions and sulfate ions.

### TECHNICAL FIELD

The present disclosure relates to a biomolecule analysis method, a biomolecule analyzing reagent, and a biomolecule analysis device.

### BACKGROUND ART

In the field of next-generation DNA sequencers, attention has been paid to a method for electrically and directly measuring the base sequence of DNA without carrying out an extension reaction or a fluorescent labeling. Specifically, research and development of a nanopore DNA sequencing method have been actively promoted. This method is a method in which a DNA strand is directly measured without using a reagent to determine a base sequence.

In the nanopore DNA sequencing method, a base sequence is measured by measuring a blockade current generated when a DNA strand passes through a pore (hereinafter referred to as “nanopore”) formed in a thin film. Since the blockade current changes depending on the difference between individual base species contained in the DNA strand, the base species can be sequentially identified by measuring the amount of the blockade current. In this method, unlike a method in which a fluorescently labeled substrate is incorporated and analyzed by the extension activity of an enzyme, information of a DNA strand is directly acquired. Therefore, the read base length is not limited to the extension activity of the enzyme, and in principle, a long strand of DNA can be decoded, and modification to the DNA strand can also be directly decoded.

A biomolecule analysis device used for analyzing DNA in the nanopore DNA sequencing method generally includes a first liquid tank and a second liquid tank, a thin film, a first electrode and a second electrode. Each of the first liquid tank and the second liquid tank are filled with an electrolyte solution. The thin film partitions the first liquid tank and the second liquid tank. The first electrode is provided in the first liquid tank. The second electrode is provided in the second liquid tank. The biomolecule analysis device can also be configured as an array device. The array device refers to a device including a plurality of sets of liquid chambers partitioned by the thin film. For example, the first liquid tank is a shared tank, and the second liquid tank is divided into a plurality of individual tanks. In this case, an electrode is disposed in each of the shared tank and the individual tanks.

In this configuration, when a voltage is applied between the first liquid tank and the second liquid tank, an ionic current (baseline current) corresponding to the nanopore diameter flows through the nanopore. A potential gradient corresponding to the applied voltage is formed in the nanopore. When a biomolecule such as DNA is introduced into the first liquid tank, the biomolecule is transported to the second liquid tank via the nanopore according to diffusion and potential gradient. At this time, the inside of the biomolecule is analyzed according to the blockade rate of each nucleic acid blocking the nanopore. A biomolecule analysis apparatus includes a measurement unit that measures an ionic current (blockade signal) flowing between the first and second electrodes provided in the biomolecule analysis device. The measurement unit acquires sequence information of the biomolecule based on a value of the measured ionic current (blockade signal).

The blockade signal generated by blocking of the nanopore by DNA is generated depending on the blockade in the nanopore and substances retained at the inlet and outlet of the nanopore. Therefore, the resolution of DNA strands is determined by the resistance in this nanopore and resistance components at the inlet and outlet of the nanopore. Here, it is assumed that DNA as a strand is analyzed in nanopore DNA sequencing. However, an enzyme may be placed at the nanopore inlet to control a nanopore passage speed of the DNA, and this enzyme may also be one of the blockade resistant components. In addition, the resistance components at the inlet and the outlet of the nanopore are also changed by coil-shaped DNA at the inlet and the outlet of the nanopore. The change in the amount of the blockade signal caused by these resistance components hinders the detection of the amount of change in the current signal according to the type of base.

To avoid the change in the amount of the blockade signal caused by the resistance components, the contemplated method is a method including: degrading DNA into nucleotides; and determining the base species one base at a time based on the blockade amount of the nucleotides, instead of analyzing the chain DNA.

NPL 1 describes that the degree of separation of nucleotides is measured with a KCl solution using a bio-nanopore. Meanwhile, NPL 2 describes that the degree of separation of signals is confirmed by allowing nucleotides to pass through a nanopore.

### CITATION LIST

### Non Patent Literatures

NPL 1: Clark, J. et al., Nature Nanotechnology, DOI: 10.1038/NNANO. 2009.12
NPL 2: Yang, H. et al., J. Phys. Chem. B 2018, 122, 7929-7935

### SUMMARY OF INVENTION

### Technical Problem

In the meantime, since the solid-state nanopore has a high affinity for LSI, high integration is expected. Further, since the solid-state nanopore can prolong the device storage period, there is a high expectation for realizing cost reduction. In NPL 1, measurement using a bio-nanopore is described, and no solid-state nanopore is examined.

In NPL 2, it is expected that it is difficult to convert a blockade signal when a single base passes through a nanopore into a species of nucleotide, because the degree of separation of the blockade signals as a result of measuring nucleotides in the solid-state nanopore is low.

Therefore, the present disclosure provides a technique for improving the ability of discriminating biomolecules in a solid-state nanopore.

### Solution to Problem

A biomolecule analysis method of the present disclosure includes: preparing a biomolecule analysis device that includes a thin film having a nanopore with a diameter in a range of ±20% of a diameter of a biomolecule, a first liquid tank and a second liquid tank separated by the thin film, a first electrode disposed in the first liquid tank, a second electrode disposed in the second liquid tank, and a biopolymer degradation mechanism that degrades a biopolymer into the biomolecule; degrading a biopolymer into the biomolecule in the biopolymer degradation mechanism; and applying a voltage between the first electrode and the second electrode in a state where a measurement solution is enclosed in the first liquid tank and the second liquid tank, and measuring a current flowing between the first electrode and the second electrode, in which the measurement solution contains ammonium ions and sulfate ions.

Additional characteristics related to the present disclosure will be apparent from the description of the present specification and the attached drawings. Aspects of the present disclosure are achieved and realized by elements, combinations of various elements, and aspects of the following detailed description, and the appended scope of claims. The description herein is merely exemplary and does not limit the scope of claims or application examples of the present disclosure in any sense.

### Advantageous Effects of Invention

According to the technique of the present disclosure, the ability of discriminating biomolecules in solid-state nanopores can be improved. Problems, configurations, and effects other than those described above will be clarified by the description of embodiments below.

### BRIEF DESCRIPTION OF DRAWINGS

FIG. 1 is a flowchart illustrating a biomolecule analysis method.

FIG. 2A is a schematic cross-sectional view illustrating a configuration of a biomolecule analysis apparatus.

FIG. 2B is a schematic diagram illustrating a pretreatment flow.

FIG. 3 is a schematic cross-sectional view illustrating another configuration of the biomolecule analysis apparatus.

FIG. 4A is a graph showing results of Experimental Example 1.

FIG. 4B is a graph showing results of Experimental Example 1.

FIG. 5A is a graph showing results of Experimental Example 2.

FIG. 5B is a graph showing results of Experimental Example 2.

FIG. 5C is a graph showing results of Experimental Example 2.

FIG. 5D is a graph showing results of Experimental Example 2.

FIG. 6A shows graphs showing results of Experimental Example 3.

FIG. 6B is a graph showing results of Experimental Example 3.

### DESCRIPTION OF EMBODIMENTS

Hereinafter, embodiments of the present disclosure will be described with reference to the drawings. The attached drawings illustrate specific embodiments according to the principles of the present disclosure. The drawings are intended to promote understanding of the technique of the present disclosure, and should never be used to construe the technique of the present disclosure narrowly.

In the present disclosure, the term “biomolecule” refers to, for example, a nucleotide constituting a nucleic acid (such as DNA, RNA, or PNA) and an analog thereof, and refers to an amino acid constituting a protein and a modified product thereof, and may be a natural product or an artificial product.

In the present disclosure, the term “analysis” of biomolecules refers to characteristic analysis of biomolecules. Characterization of biomolecules includes, for example, analysis of the sequence order of monomers of nucleic acids (sequence determination), determination of the length of nucleic acids, detection of single nucleotide polymorphisms, and detection of structural polymorphisms (copy number polymorphism, insertion, deletion, etc.) in biomolecules.

### [Biomolecule Analysis Method]

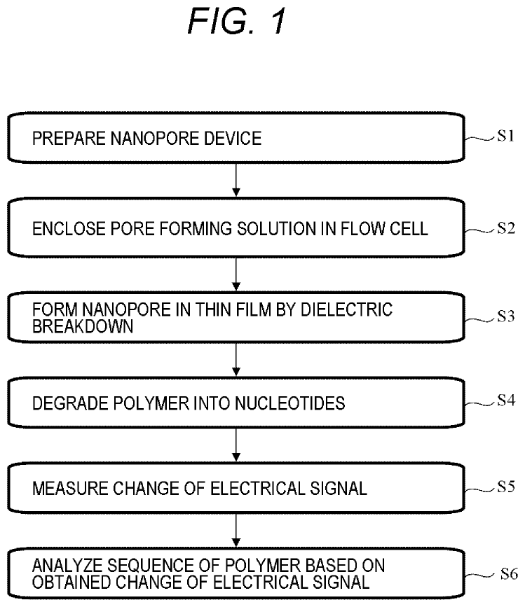

FIG. 1 is a flowchart illustrating a biomolecule analysis method according to a first embodiment.

Hereinafter, a case where a nucleic acid is used as an example of a biopolymer and a nucleotide is measured as an example of a biomolecule may be described.

### (Step S1: Preparation of Biomolecule Analysis Apparatus)

In step S1, an operator prepares a biomolecule analysis apparatus including a solid-state nanopore device (a biomolecule analysis device). Specifically, for example, the nanopore device includes a thin film on which a nanopore is to be formed, and can be produced by installing the thin film in the flow cell. As a result, liquid tanks are formed on both sides of the thin film. A first electrode is disposed in one liquid tank (first liquid tank), and a second electrode is disposed in the other liquid tank (second liquid tank). A power source for applying a voltage is connected between the first electrode and the second electrode of the nanopore device. Further, the operator installs an ammeter that measures the current between the first electrode and the second electrode. Thus, the biomolecule analysis apparatus is prepared.

The biomolecule analysis apparatus of the present embodiment is provided with a biopolymer degradation unit disposed on the upstream side of the thin film on which the nanopore is formed. The biopolymer degradation unit has a flow path through which the biopolymer flows, and degrades the biopolymer into monomers (biomolecule) in the flow path. Disposed in the flow path is a substance capable of degrading the biopolymer, for example, a biopolymer-degrading enzyme (such as exonuclease or an analog thereof), a highly concentrated acid (pyrophosphoric acid or hydrochloric acid), or the like. Alternatively, the biopolymer degradation unit may be configured such that the flow path is irradiated with a laser beam capable of degrading the biopolymer.

### (Step S2: Enclosing of Nanopore Forming Solution)

In step S2, the operator encloses a nanopore forming solution (electrolyte solution) for opening a nanopore in the first liquid tank and the second liquid tank from a supply port of the flow cell.

### (Step S3: Opening of Nanopore)

In step S3, the operator drives the power source to apply a voltage for opening a nanopore between the first electrode and the second electrode so as to form a nanopore with a predetermined diameter in a thin film by dielectric breakdown.

### (Step S4: Degradation of Biopolymer)

In step S4, the operator drives a power source to apply a voltage for analysis between the first electrode and the second electrode, and encloses a measurement solution containing a biopolymer (nucleic acid) to be measured from a sample injection port. Thereafter, the biopolymer migrates in the flow path and is degraded into biomolecules (nucleotides) when passing through the biopolymer degradation unit, and the biomolecules (nucleotides) are introduced into the first liquid tank.

### (Step S5: Measurement)

In step S5, the operator uses an ammeter to measure a change of an electrical signal (current value) from the first electrode and the second electrode.

### (Step S6: Analysis of Biomolecule)

In step S6, the operator uses a computer device to analyze the biomolecule based on the change of the electrical signal. When the biomolecule passes through the nanopore, the electrical signal changes according to the monomer type (base type). Thus, the sequence can be determined according to the pattern of the electrical signal. Details of the method are disclosed in the literature (A. H. Laszlo, et al., Nature Biotechnology 32, 829, 2015).

### (Nanopore Diameter and Electrolyte Solution)

The present inventors have intensively studied on an electrolyte solution used for analysis of nucleotides. As a result, it has been unexpectedly found that, in a case of using a measurement solution containing ammonium ions as cations of an electrolyte and sulfate ions as anions, signals derived from nucleotides cannot be confirmed when the nanopore diameter is 1.4 nm or more, whereas when the nanopore diameter is smaller than 1 nm, signals derived from nucleotides start to be clearly confirmed, and the blockade amounts derived from four types of nucleotides constituting a biopolymer are clearly different. Hence, it has been found that the ability of discriminating biomolecules can be improved by using a nanopore with a diameter of ±20% or less of a diameter of a biomolecule (specifically, for example, a diameter of 1 nm or less) and using an electrolyte solution containing ammonium ions and sulfate ions as a measurement solution.

Therefore, in the biomolecule analysis method of the present embodiment, the measurement solution (may be simply referred to hereinafter as “electrolyte solution”) contains ammonium ions (NH₄⁺) as cations of the electrolyte and contains sulfate ions (SO₄²⁻) as anions. That is, the electrolyte of the electrolyte solution generates ammonium ions as cations and sulfate ions as anions. Both the nanopore forming solution and the measurement solution can also contain ammonium ions and sulfate ions.

As the electrolyte (salt) that generates ammonium ions and sulfate ions, for example, ammonium sulfate can be used. As the electrolyte, sulfate and ammonium salt that ionize in a solvent can also be used. Examples of the sulfate include magnesium sulfate, sodium sulfate, potassium sulfate, copper sulfate, and iron sulfate. Examples of the ammonium salt include ammonium chloride and ammonium carbonate.

To ensure electrical conductivity, the electrolyte solution may contain ions other than ammonium ions and sulfate ions. The cations can be selected from, for example, any metal ion. Note that, for example, monovalent metal ions such as potassium ions may promote the bond dissociation of dangling bonds on the SiN surface. In addition, divalent metal ions have a certain effect in reducing noise superimposed on the baseline current, but the presence of the divalent metal ions at a high concentration results in reaction with other ions, leading to precipitation. Therefore, when cations other than ammonium ions are contained in the electrolyte solution, it is necessary to appropriately adjust the type and concentration thereof. The anions can be selected depending on the compatibility with the electrode material. For example, when silver halide is used as the electrode material, the anions contained in the electrolyte solution can be halide ions (chloride ions, bromide ions, iodide ions). Alternatively, the anions may be organic anions represented by glutamic acid ion or the like.

Hence, an electrolyte (salt) other than ammonium sulfate or sulfate and ammonium salt can coexist in the electrolyte solution. Examples of the electrolyte include KCl, NaCl, LiCl, and CsCl. When platinum or Au is used for the electrode, ferricyanide or ferrocyanide may coexist. When a molecular motor is used as one means for arbitrarily controlling the transportation of the biomolecule, a substrate and a buffer suitable for driving the molecular motor coexist in the electrolyte solution in the first liquid tank. In order to stabilize the biomolecule, a buffer can be mixed. In general, MgSO₄, MgCl₂, Tween (registered trademark), HEPES, Tris-HCl, EDTA, glycerol, or the like can be mixed as a buffer.

As a solvent of the electrolyte solution, it is possible to use a solvent which can stably disperse the biomolecule, does not dissolve an electrode, and does not inhibit electron transfer with the electrode. Examples of the solvent of the electrolyte solution include water, alcohols (such as methanol, ethanol, and isopropanol), acetic acid, acetone, acetonitrile, dimethylformamide, and dimethylsulfoxide. When a nucleic acid as the biomolecule is used as an object to be measured, water is typically used.

The lower limit of the concentration of the electrolyte is provided, whereby a signal-to-noise ratio (SNR) can be improved. Specifically, for example, the lower limit of the concentration of the electrolyte can be set to 0.01 M. Meanwhile, there is no requirement for hindering the upper limit of the concentration of the electrolyte, and the concentration of the electrolyte can be allowed up to a saturation concentration. That is, when the electrolyte solution contains only ammonium sulfate as the electrolyte (salt), the concentration of ammonium sulfate can be set to 0.01 M or more and a saturation concentration or less. The concentration of ammonium sulfate can be set to 0.01 M or more and 4 M or less, or 0.01 M or more and 2 M or less, in some situations.

When the electrolyte solution contains ammonium sulfate and another salt as the electrolyte (salt), the ratio of the ammonium sulfate concentration to the total salt concentration can be set to 5% or more and less than 100%. In some situations, the ratio of the ammonium sulfate concentration to the total salt concentration can be set to 25% or more and less than 100%, or 50% or more and less than 100%.

When the electrolyte solution contains sulfate, ammonium salt, and another salt as an electrolyte, the ratio of the sulfate ion concentration to the total anion concentration can be set to 5% or more and less than 100%. In some situations, the ratio of the sulfate ion concentration to the total anion concentration can be set to 25% or more and less than 100%, or 50% or more and less than 100%. The ratio of the ammonium ion concentration to the total cation concentration can be set to 5% or more and less than 100%. In some situations, the ratio of the ammonium ion concentration to the total cation concentration can be set to 25% or more and less than 100%, or 50% or more and less than 100%.

The nanopore can be formed not only by dielectric breakdown but also by preliminary microfabrication or processing using a TEM device. In this case, in step S1 described above, the operator assembles the nanopore device using the thin film in which the nanopore is preliminarily formed, and does not perform steps S2 and S3.

The measurement can be performed using the nanopore forming solution without replacing the nanopore forming solution used in steps S2 and S3 with the measurement solution. In this case, the nanopore forming solution contains ammonium ions (NH₄⁺) as cations of the electrolyte and contains sulfate ions (SO₄²⁻) as anions, similarly to the measurement solution described above. Meanwhile, the biomolecule can be more accurately analyzed by replacing the nanopore forming solution with the measurement solution that is more suitable for the biomolecule as in step S4 described above.

### (Conclusion)

As described above, in the biomolecule analysis method according to the present embodiment, a nanopore device is used, which has a nanopore with a diameter of ±20% or less of a diameter of a biomolecule, and the measurement solution contains ammonium ions as cations and sulfate ions as anions. Otherwise, the biomolecule analysis method can be carried out using the same device, steps, and conditions as those in the conventional method. The use of the measurement solution makes it possible to reduce the variation in the amount of blockade signal derived from the biomolecule (nucleotide) passing through the nanopore, and to determine the type of biomolecule (nucleotide) passing through the nanopore at a high signal-to-noise ratio. In addition, the use of a nanopore with a diameter of ±10% or less of a diameter of a biomolecule makes it possible to further reduce the variation in the amount of the blockade signal derived from the biomolecule. In particular, the nucleotide is measured using a nanopore with a diameter of 1 nm or less, as a result of which the blockade signal derived from the nucleotide can be detected. At this time, the dispersion of the amounts of blockade signals derived from various nucleotides is reduced, and thus it is easy to determine the types of nucleotides.

### [Biomolecule Analyzing Reagent and Biomolecule Analysis Device]

The biomolecule analyzing reagent of the present disclosure can be provided as a consumable for measurement, and contains the electrolyte of the electrolyte solution described above as a component. That is, when the biomolecule analyzing reagent in a biomolecule analysis kit is formed into a solution, the solution contains ammonium ions as cations and sulfate ions as anions. The biomolecule analyzing reagent is used as a measurement reagent (a nanopore forming reagent or a measurement reagent, in some situations). The biomolecule analysis device (nanopore device) is provided as a consumable for measurement, and includes a nanopore having the above-described dimension as a component. The biomolecule analysis device can be provided in which a nanopore of 1 nm or less is preliminarily formed. Alternatively, the biomolecule analysis device can be provided with only a thin film, and a nanopore of 1 nm or less is formed after being set in the biomolecule analysis apparatus immediately before measurement.

The biomolecule analysis kit of the present disclosure can be provided together with a manual describing the procedure and amount of use. The biomolecule analyzing reagent may be provided in a ready-to-use state (the nanopore forming solution and the measurement solution described above). Alternatively, the biomolecule analyzing reagent may be provided as a concentrated solution for dilution with an appropriate solvent during use. Alternatively, the biomolecule analyzing reagent may be in a solid state for reconstitution with an appropriate solvent during use (e.g. powder). Those skilled in the art can understand the form and preparation of such a biomolecule analyzing reagent. The biomolecule analysis device may be provided in contact with the biomolecule analyzing reagent, or may be brought into contact with the reagent after being set in the biomolecule analysis apparatus immediately before measurement.

The nanopore forming reagent is used when a voltage is applied between the two liquid tanks formed on both sides of the thin film to form the nanopore by dielectric breakdown. The measurement reagent is used in passing the biomolecule through the nanopore and measuring a current (blockade current) flowing through the nanopore. The concentration of the electrolyte of the nanopore forming reagent and the concentration of the electrolyte of the measurement reagent may be identical or different. The nanopore forming reagent may be a reagent having a conventional composition. These reagents and the device may be provided to a user as a set of the nanopore forming reagent, the measurement reagent, and the device, or may be separately provided.

### (Conclusion)

As described above, the biomolecule analysis kit according to the present embodiment includes the biomolecule analyzing reagent. When the reagent is used as the measurement solution, ammonium ions are generated as cations, and sulfate ions are generated as anions. In the thin film of the biomolecule analysis device, a 1 nm nanopore can be produced by the nanopore forming solution. The use of the biomolecule analysis kit makes it possible to reduce the variation in the amount of blockade signal derived from the biomolecule (nucleotide) passing through the nanopore, and to determine the type of biomolecule (nucleotide) passing through the nanopore at a high signal-to-noise ratio.

### [Biomolecule Analysis Apparatus]

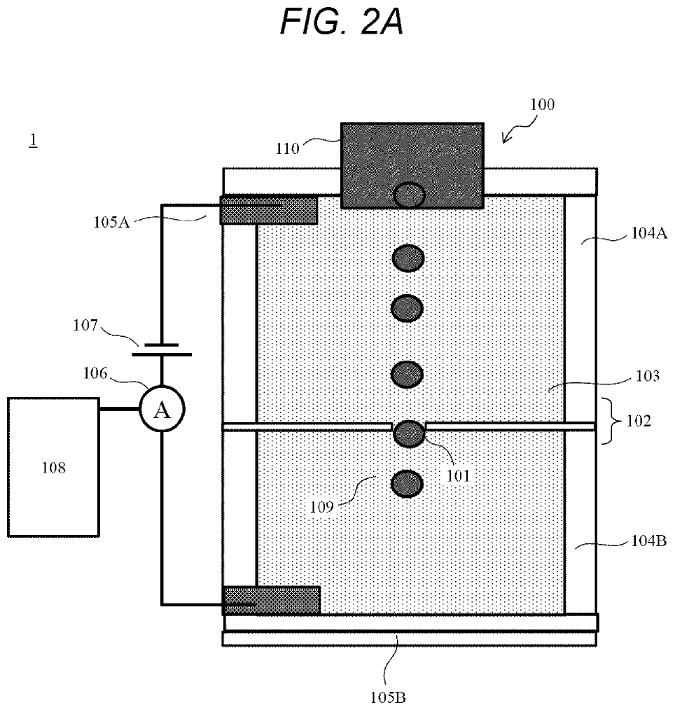

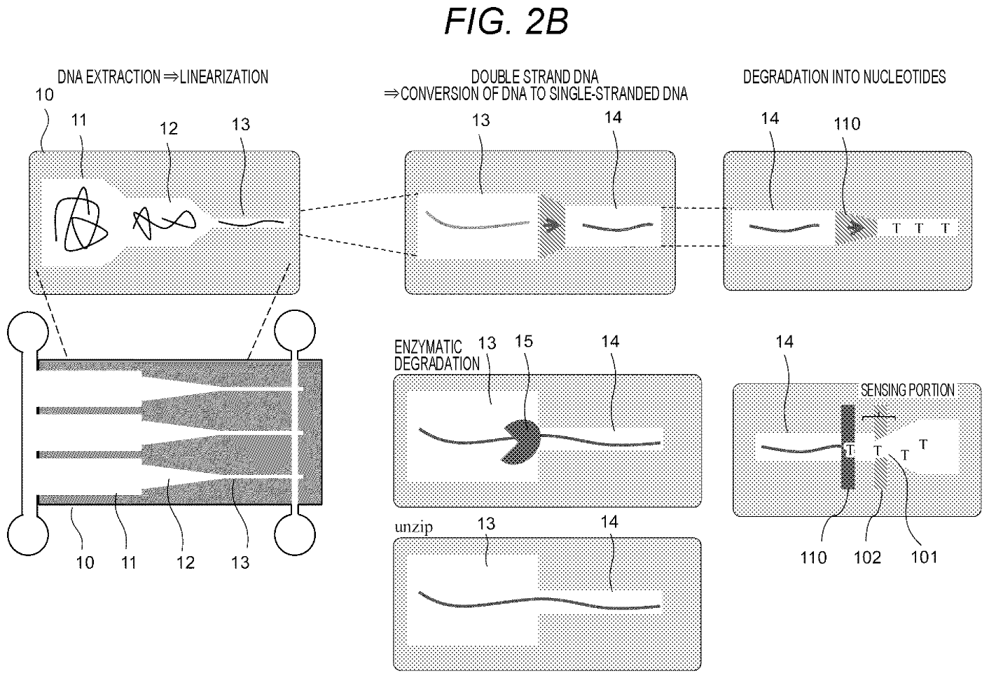

FIG. 2A is a schematic cross-sectional view illustrating a configuration of a biomolecule analysis apparatus 1 according to the first embodiment. The biomolecule analysis apparatus 1 is an apparatus that degrades the biopolymer in a pretreatment mechanism (biopolymer degradation mechanism) and measures an ionic current by a blockade current method to measure characteristics of the biomolecule formed by the degradation.

As illustrated in FIG. 2A, the biomolecule analysis apparatus 1 includes a nanopore device 100, an ammeter 106, a power source 107, a computer 108, and a biopolymer degradation mechanism 110. The nanopore device 100 includes a thin film 102 in which a nanopore 101 is formed, a first liquid tank 104A, a second liquid tank 104B, a first electrode 105A, and a second electrode 105B. The first liquid tank 104A and the second liquid tank 104B are disposed to be in contact with the thin film 102 with the thin film 102 interposed therebetween. An electrolyte solution 103 is filled in the first and second liquid tanks. The first electrode 105A is provided in the first liquid tank 104A. The second electrode 105B is provided in the second liquid tank 104B.

In the nanopore device 100 of FIG. 2A, the nanopore 101 is formed in the thin film 102, and a biomolecule 109, which is a product degraded by the biopolymer degradation mechanism 110, is sequentially introduced into the nanopore 101.

The biomolecule 109 may be an object to be measured that changes electrical characteristics, particularly a resistance value, when passing through the nanopore. The biomolecule 109 is typically a nucleotide constituting single-strand DNA, double-strand DNA, RNA, PNA (peptide nucleic acid), an amino acid constituting a protein, or a modified product thereof (e.g. a nucleotide analogue). When the nanopore device 100 analyzes a nucleotide sequence constituting the biopolymer, the biomolecule 109 needs to pass through the nanopore according to the sequence. As a means for allowing the biomolecule 109 to pass through the nanopore 101, transportation by electrophoresis can be adopted, and the means may be a solvent flow generated by a pressure potential difference or the like.

The electrolyte solution 103 is the nanopore forming solution or the measurement solution as described above. The electrolyte solution 103 has, for example, a volume of microliter order or milliliter order.

The power source 107 applies a predetermined voltage between the first electrode 105A and the second electrode 105B. When a voltage is applied between the first electrode 105A and the second electrode 105B, a potential difference occurs between both surfaces of the thin film 102 in which the nanopore 101 is formed. The biomolecule 109 dissolved in the first liquid tank 104A (cis tank) located on the upper side is migrated toward the lower second liquid tank 104B (trans tank) located on the lower side.

The ammeter 106 measures an ionic current (blockade signal) flowing between the first electrode 105A and the second electrode 105B, and outputs the measurement value to the computer 108. The ammeter 106 includes an amplifier that amplifies the current flowing between the electrodes by application of a voltage and an analog-to-digital converter (ADC) (not illustrated). A detection value which is an output of the ADC is output to the computer 108.

The computer 108 controls voltages applied to the first electrode 105A and the second electrode 105B by the power source 107. Further, the computer 108 analyzes the biomolecule 109 based on the detection value of the current from the ammeter 106. More specifically, the computer 108 acquires sequence information of the biomolecule 109 based on the value of the ionic current (blockade signal).

The most effective nanopore measurement method in which the technique of the present disclosure exerts the effect is the method for measuring the blockade current as described above. The following methods may be added to compensate for information. One of the methods is a method for providing another pair of electrodes in the vicinity of the nanopore in addition to the first electrode 105A and the second electrode 105B, applying a voltage between the pair of electrodes, and measuring a change in tunnel current generated when the biomolecule passes. Alternatively, there is a method for providing an FET device on a nanopore membrane and measuring a signal change of a transistor obtained by the device. There is a method for measuring Raman scattered light by forming gold or silver bowties in the vicinity of the nanopore or arranging gold or silver particle dimers and then emitting light to generate a near field. It is also possible to measure optical signals such as absorption, reflection, and fluorescence characteristics of light emitted to the vicinity of the nanopore.

The computer 108 typically includes an ionic current measurement device, an analog-digital converter, a data processing device, a data display output device, and an input/output auxiliary device. The ionic current measurement device is equipped with a current-voltage converting type high-speed amplifying circuit. The data processing device is equipped with an arithmetic device, a temporary storage device, and a non-volatile storage device. Covering the nanopore device 100 with a Faraday cage enables external noise to be reduced.

As illustrated in FIG. 2A, the ammeter 106, the power source 107, and the computer 108 may not be separate members from the nanopore device 100, but may be integrated with the nanopore device 100.

Hereinafter, a method for producing the above-described biomolecule analysis apparatus 1 will be described. It is known in the art that the basic configuration itself of the biomolecule analysis apparatus is used for analyzing the biomolecule with then so-called blockade current system. The components of the biomolecule analysis apparatus can also be easily understood by those skilled in the art. For example, specific devices are disclosed in U.S. Pat. No. 5,795,782 and “Scientific Reports 4, 5000, 2014, Akahori, et al.”, “Nanotechnology 25(27): 275501, 2014, Yanagi, et al.”, “Scientific Reports, 5, 14656, 2015, Goto, et al.”, and “Scientific Reports 5, 16640, 2015”.

The thin film 102 in which the nanopore 101 is formed is a thin film (solid-state pore) made of a material that can be formed by a semiconductor microfabrication technique. Examples of the material that can be formed by the semiconductor microfabrication technique include silicon nitride (SiN), silicon oxide (SiO₂), silicon oxynitride (SiON), hafnium oxide (HfO₂), molybdenum disulfide (MoS₂), and graphene. The thickness of the thin film 102 can be set to 1 Å (angstrom) to 200 nm, optionally 1 Å to 100 nm, or 1 Å to 50 nm in some situations, and specifically, for example, about 5 nm.

The area of the thin film 102 is set to an area in which it is difficult to form two or more nanopores 101 when forming the nanopores 101 by applying a voltage, and an area with which allowable strength is provided. As an example, the area can be set to, for example, about 100 to 500 nm². The thickness of the thin film 102 is set to a thickness that can form the nanopore 101 having an effective film thickness equivalent to one base, thereby achieving DNA one base resolution. As an example, the film thickness can be set to about 7 nm or less. The thin film 102 may have a structure in which both surfaces are sandwiched by another thin film having a through-hole. In this case, the area of the thin film 102 exposed by the through-holes on both surfaces may be set as described above.

As the dimension (diameter) of the nanopore 101, an appropriate dimension can be selected according to the type of the biomolecule 109 to be analyzed. The diameter of the nanopore 101 is designed to be ±20% of the diameter of the biomolecule 109 to be measured. As an example, when nucleotides constituting DNA are measured, the dimension of the nanopore 101 can be, for example, 0.7 nm to 1.0 nm.

The depth of the nanopore 101 can be adjusted by adjusting the thickness of the thin film 102. The depth of the nanopore 101 can be set to equal to or more than 2 times the biomolecule 109 (monomer unit), and can be set to equal to or more than 3 times, or equal to or more than 5 times, in some situations. For example, when the biomolecule 109 is composed of nucleotide, the depth of the nanopore 101 can be set to a size of 3 or more bases, for example, about 1 nm or more. The shape of the nanopore 101 is basically circular, and can also be elliptical or polygonal.

In the case of an array-type apparatus configuration including a plurality of thin films 102 having nanopores 101, the thin films 102 having nanopores 101 can be regularly arranged. The interval at which the plurality of thin films 102 is arranged can be set to 0.1 μm to 1 mm or 1 μm to 700 μm depending on the electrodes to be used and the capabilities of the electrical measurement system.

The method for forming the nanopore 101 in the thin film 102 is not particularly limited. For example, electron beam irradiation by a transmission electron microscope (TEM) or the like, and dielectric breakdown by application of a voltage (pulse voltage or the like) can be used. The method described in “Itaru Yanagi et al., Sci. Rep. 4, 5000 (2014)” or “A. J. Storm et al., Nat. Mat.2 (2003)” can be used as the method for forming the nanopore 101, for example.

When a voltage is applied from the power source to the electrodes provided in the two upper and lower liquid tanks, an electric field is generated in the vicinity of the nanopore. The biomolecule that is negatively charged in the liquid passes through the nanopore. At that time, the above-mentioned blockade current Ib flows.

The first liquid tank 104A and the second liquid tank 104B can store the measurement solution in contact with the thin film 102. The first liquid tank 104A and the second liquid tank 104B can be appropriately provided with a material, a shape, and a size that do not affect the measurement of the blockade current. The measurement solution is injected so as to come into contact with the thin film 102 that partitions the first liquid tank 104A and the second liquid tank 104B.

The first electrode 105A and the second electrode 105B can be produced with a material capable of causing an electron transfer reaction (Faraday reaction) with the electrolyte in the measurement solution. The first electrode 105A and the second electrode 105B are typically produced with silver halide or alkali silver halide. From the viewpoint of potential stability and reliability, it is possible to use silver or silver-silver chloride.

The first electrode 105A and the second electrode 105B may be produced with a material serving as a polarization electrode. The first electrode 105A and the second electrode 105B may be produced with, for example, gold or platinum. In this case, a substance capable of assisting the electron transfer reaction, for example, potassium ferricyanide or potassium ferrocyanide, can be added to the measurement solution in order to secure a stable ionic current. Alternatively, it is also possible to immobilize a substance capable of carrying out the electron transfer reaction, for example, ferrocenes, on the surface of the polarization electrode.

The structures of the first electrode 105A and the second electrode 105B may be entirely made of the above-described material, or the surface of a base material (copper, aluminum, etc.) may be coated with the above-described material. The shapes of the first electrode 105A and the second electrode 105B are not particularly limited. It can be adopted that the shapes of the first electrode 105A and the second electrode 105B has a large surface area in contact with the measurement solution. The first electrode 105A and the second electrode 105B are joined to wiring lines, and an electrical signal is transmitted to a measurement circuit (ammeter 106).

The biomolecule analysis apparatus 1 includes the above configuration as an element. The nanopore-type biomolecule analysis apparatus 1 as described above can be provided together with a manual describing the procedure and amount of use. Such forms and preparations can be understood by those skilled in the art. Similarly, the nanopore device 100 may be provided in a state in which the nanopore is formed in a ready-to-use state, or may be provided in a state in which the nanopore is formed in the providing destination.

### (Conclusion)

As described above, the biomolecule analysis apparatus according to the present embodiment includes the biopolymer degradation mechanism, and the biomolecule (nucleotide) as a degradation product of the biopolymer (nucleic acid) is transported to the liquid tank located on the upper portion of the thin film. Further, the electrolyte solution sealed on both sides of the thin film contains ammonium ions as cations and sulfate ions as anions. Furthermore, the diameter of the nanopore through which the biomolecule (nucleotide) to be measured passes is adjusted to ±20% of the diameter of the biomolecule (nucleotide). Consequently, it is possible to reduce the variation in the amount of the blockade signal derived from the biomolecule passing through the nanopore. Thereby, it is possible to determine the type of the passing biomolecule at a high signal-to-noise ratio.

[Flow from Pretreatment to Measurement]

The biopolymer can be pretreated before introducing the biopolymer into the biopolymer degradation mechanism 110. Hereinafter, the pretreatment step will be described by taking a case where the biopolymer is DNA as an example. As the pretreatment, for example, DNA is linearized and single-stranded.

FIG. 2B is a schematic diagram illustrating a flow until measurement becomes possible with the nanopore after the DNA polymer is pretreated and degraded. The upper left part of FIG. 2B illustrates the flow of linearization of DNA on a pretreatment chip 10. The pretreatment chip 10 includes a sample collection unit 11, a micro-flow path 12, and a nano-flow path 13, which are continuously connected to form one flow path. In the sample collection unit 11, for example, DNA extracted from cells is collected. The extracted DNA has a tertiary structure and is in a coiled state (Gaussian coil). The DNA passes through the micro-flow path 12, and thus the DNA is stretched to some extent, simultaneously with the removal of contaminants. Thereafter, the DNA passes through the nano-flow path 13 (e.g. submicron order), and thus the DNA is changed to single-stranded DNA. As illustrated in the lower left part of FIG. 2B, a plurality of flow paths (three in FIG. 2B) is provided in parallel on the pretreatment chip 10.

The upper center of FIG. 2B illustrates the flow of conversion of DNA to single-stranded DNA. For example, as illustrated in the middle of the center of FIG. 2B, the conversion of DNA to single-stranded DNA can be conducted by a reaction using a single-strand degrading enzyme 15 disposed in the nano-flow path 13. The single-stranded DNA passes through a flow path 14 of several nanometers, and is introduced into the biopolymer degradation mechanism 110. Alternatively, as illustrated in the lower center part of FIG. 2B, DNA is allowed to pass through the nano-flow path 14 having a diameter equal to or less than the double strand diameter and equal to or more than the single strand diameter using no enzyme, and thus the DNA can be converted to single-stranded DNA.

Thereafter, as illustrated on the right side of FIG. 2B, the single-stranded DNA that has passed through the nano-flow path 14 passes through the biopolymer degradation mechanism 110. Thus, the DNA is degraded from the terminal position into nucleotides. The resultant nucleotides pass through the nanopore 101, in the order of cleavage, by diffusion and electrophoresis. As described above, an enzyme that can bind to a single strand again and can degrade nucleotides is disposed in the biopolymer degradation mechanism 110. Alternatively, a high concentration of hydrochloric acid is retained. Alternatively, the biopolymer degradation mechanism 110 is configured such that the DNA can be irradiated with a laser beam.

### [Array Device]

In the nanopore device 100 of the biomolecule analysis apparatus 1 illustrated in FIG. 2A, one thin film 102 has only one nanopore 101. However, this is merely an example. It is also possible to provide an array device configured by forming a plurality of nanopores 101 in the thin film 102 and separating each region of the plurality of nanopores 101 by a partition wall. Therefore, a configuration example of the array device will be described below.

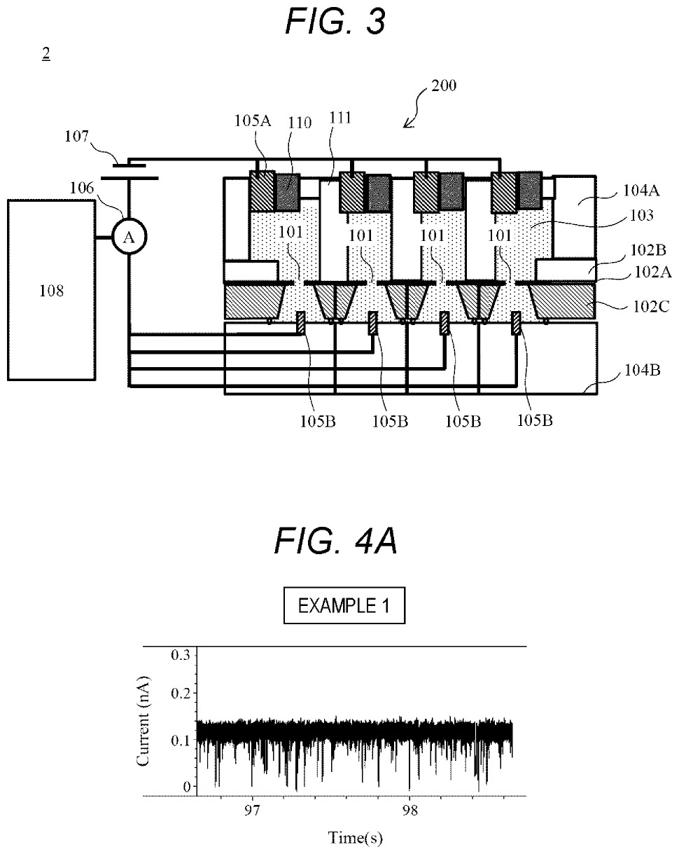

FIG. 3 is a schematic cross-sectional view illustrating a configuration of a biomolecule analysis apparatus 2. In FIG. 3, the same components as those of the biomolecule analysis apparatus 1 illustrated in FIG. 2A are denoted by the same reference numerals, and redundant description will be omitted. As illustrated in FIG. 3, the biomolecule analysis apparatus 2 is different from the biomolecule analysis apparatus 1 of FIG. 2A in terms of including a nanopore device 200, i.e. an array device.

In the nanopore device 200, the thin film 102A has a plurality of nanopores 101. The second liquid tank 104B below the thin film 102A is divided into spaces by partition walls (specifically, sidewalls of the thin film 102C). In the thin films 102B and 102C that fix the thin film 102A, through-holes are provided at positions corresponding to the nanopores 101. A plurality of spaces (individual tanks) is formed by the sidewalls of the through-holes of the thin film 102C. The second electrode 105B is provided in each of the plurality of spaces. The first liquid tank 104A is also divided into individual spaces by partition walls 111 such that respective biopolymers are not mixed, and is insulated. Therefore, the currents flowing through the nanopores 101 can be measured independently. Each of the first liquid tanks 104A is provided with an individual biopolymer degradation mechanism 110.

As the nanopore forming solution or the measurement solution (the electrolyte solution 103), the above-described solution may be used. Accordingly, the type of nucleotide passing through the nanopore can be determined with high determination accuracy. Since the biomolecule analysis apparatus 2 can perform measurement in parallel, it is possible to perform monomer sequence analysis of biomolecules with very high throughput while maintaining high analysis accuracy.

The biomolecule analysis method, the biomolecule analyzing reagent, and the biomolecule analysis device according to the present disclosure are useful, for example, in analysis of biomolecules composed of nucleic acids, and are useful in the fields such as test, diagnosis, therapy, drug discovery, and basic study using the analysis.

### EXAMPLES

Hereinafter, the technique of the present disclosure will be described in more detail using Examples. However, the technique of the present disclosure is not limited to these Examples.

### [Preparation of Biomolecule Analysis Apparatus]

In the following Examples, used is a single-pore biomolecule analysis apparatus having the configuration illustrated in FIG. 2A. First, a nanopore device was produced as follows.

According to the following procedure, a thin film was produced by a semiconductor microfabrication technique. First, Si₃N₄, polySi, and Si₃N₄ were formed in the order of thicknesses of 5 nm, 150 nm, and 100 nm, respectively, on the front surface of an 8 inch Si wafer having a thickness of 725 mm. Further, a 105-nm Si₃N₄ film was formed on the back surface of the Si wafer. The polySi as the intermediate layer may be SiO.

Next, the Si₃N₄ at the top of the front surface of the Si wafer was removed by reactive ion etching in 500 nm square. Similarly, the Si₃N₄ on the back surface of the Si wafer was removed by reactive ion etching in 1038 μm square. The back surface of a Si substrate exposed by etching was further etched with tetramethylammonium hydroxide (TMAH). During Si etching, the surface of the wafer was covered with a protective film (ProTEK (registered trademark) B3 primer and ProTEK (registered trademark) B3, manufactured by Brewer Science, Inc.) in order to prevent etching of polySi on the front surface side.

Then, after the protective film was removed, the polySi layer exposed in 500 nm square was removed with an NH₄OH solution. As a result, a partition body was obtained in which the Si₃N₄ thin film having a film thickness of 5 nm was exposed. When SiO is selected as a sacrificial layer, the thin film is exposed by etching with a BHF solution (HF:NH₄F=1:60). At this stage, the nanopore is not provided in the thin film.

The nanopore was formed by the following procedure. Before the partition body was set in a biomolecule analysis device or the like, the Si₃N₄ thin film was hydrophilized by immersing the partition body in a piranha solution (H₂SO₄: H₂O₂=3:1) for 3 minutes. After the immersion, the partition body was washed under running pure water for 5 minutes or more. Hydrophilization can also be performed under conditions of 10 W, 20 sccm, 20 Pa, and 45 sec by Ar/O₂ plasma (manufactured by Samco Inc.). Then, the partition body was set in the biomolecule analysis device. Thereafter, upper and lower liquid tanks sandwiching the thin film were filled with a nanopore forming solution, and an electrode was introduced into each of the liquid tanks. As the electrode, a silver-silver chloride electrode was used. Water was used as a solvent of the nanopore forming solution.

The voltage is applied not only when the nanopore is formed, but also when the ionic current flowing through the nanopore is measured after the nanopore is formed. Here, the liquid tank located on the lower side is referred to as a cis tank, and the liquid tank located on the upper side is referred to as a trans tank. A voltage Vcis to be applied to the electrode on the cis tank side is set to 0 V. A voltage Vtrans is applied to the electrode on the trans tank side. The voltage Vtrans is generated by a pulse generator (for example, 41501B SMUAND Pulse Generator Expander, manufactured by Agilent Technologies, Inc.).

The current value after the pulse application can be read by an ammeter (for example, 4156B PRECISION SEMICONDUCTOR ANALYZER manufactured by Agilent Technologies, Inc.). The current value condition (threshold current) is selected in accordance with the diameter of the nanopore formed before the application of the pulse voltage. The desired diameter can be obtained while sequentially increasing the diameter of the nanopore.

The diameter of the nanopore can be estimated from the ionic current value. Condition selection criteria are as shown in Table 1.

TABLE 1
Voltage application conditions

| | Nanopore diameter before | | |
|---|---|---|---|
| | pulse voltage application | | |
| | Non-opening | | |
| | to Φ1.0 nm | to Φ1.4 nm | to Φ1.5 nm |
| Applied voltage (V_{cis}) [V] | 5 | 3 | 2.5 |
| Initial application time [s] | 0.01 | 0.01 | 0.001 |
| Threshold current | 0.2 nA/0.4 V | 0.5 nA/0.1 V | 0.6 nA/0.1 V |

Here, n-th pulse voltage application time tₙ (where n is an integer of more than 2) is determined by the following expression.

$${t}_{n} = 1 {0}^{- 3 + ( 1 / 6 ) ( n - 1 )} - 1 {0}^{- 3 + ( 1 / 6 ) ( n - 2 )} For n > 2 \qquad [ Expression 1 ]$$

### Experimental Example 1: Change of Nanopore Diameter

### Example 1

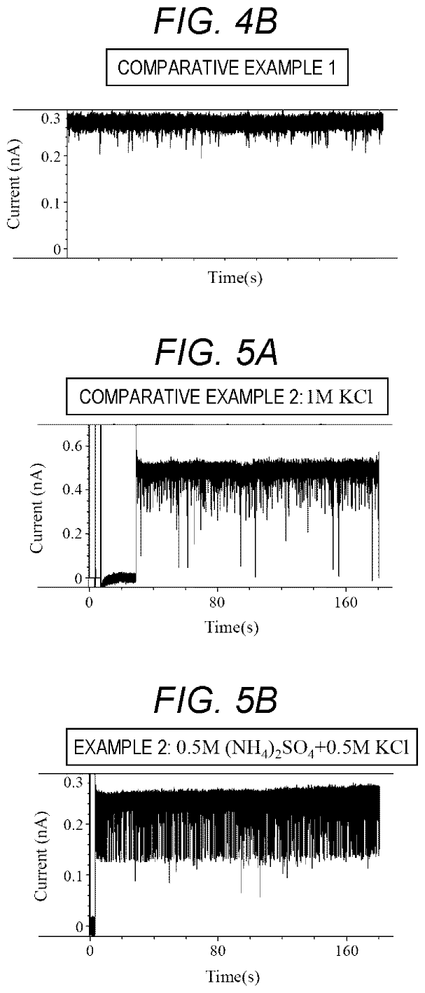

In Example 1, a nanopore was formed using a 0.5 M (NH₄)₂SO₄+0.5 M KCl+10 mM Tris-HCl solution (pH 7.5) as a nanopore forming solution. The pore conductivity obtained by this nanopore forming solution was 1.94 nS. The diameter of the nanopore converted as an effective film thickness of 3.5 nm was 0.94 nm. Here, the effective film thickness was determined on the basis of the base current value dependency of the blockade amount when dsDNA was measured, assuming that the effective diameter of dsDNA was 2.5 nm. Thereafter, the nanopore forming solution was discharged. The inside of the cis tank was replaced with a 0.2 M (NH₄)₂SO₄+1×enzyme buffer (pH 7.5)+Tween (registered trademark) 20 solution (without Mg) as a measurement solution. The inside of the trans tank was replaced with a 0.5 M (NH₄)₂SO₄+0.5 M MgSO₄+10 mM Tris-HCl solution (pH 7.5) as a measurement solution. After the replacement with the measurement solution, the time change of a baseline current was measured. Thereafter, 100 μM dNTP was added, and the time change of an ionic current (blockade signal amount) was measured. The results are illustrated in FIG. 4A.

### Comparative Example 1

In Comparative Example 1, the amount of the blockade signal of dNTP-derived signal when a nanopore having a size different from that of Example 1 was formed was compared with that in the case of Example 1. Specifically, a nanopore was formed such that the conductivity obtained after the formation of the nanopore by the nanopore forming solution was 5.74 nS. The diameter of the nanopore calculated as an effective film thickness of 3.5 nm was 1.72 nm. The time change of a baseline current and the time change in an ionic current after addition of 100 μM dNTP were measured in the same manner as in Example 1 except that the diameter of the nanopore was changed. The results are illustrated in FIG. 4B.

### (Results)

FIG. 4A is a graph showing a baseline current after addition of 100 μM dNTP in Example 1. As illustrated in FIG. 4A, in Example 1, the obtained distribution of the amount of a blockade signal derived from dNTP was Ib to 90 pA (LPF 2 kHz). A clear signal that was considered to be derived from dNTP was obtained.

FIG. 4B is a graph showing an ionic current after addition of 100 μM dNTP in Comparative Example 1. As illustrated in FIG. 4B, in Comparative Example 1, the obtained distribution of the amount of the blockade signal derived from dNTP was up to the range of Ib to 60 pA (LPF 2 kHz). Only the signal considered to be derived from base current noise was obtained. From this, it is found that that when an ammonium sulfate solution is used as the measurement solution as in Example 1 and the diameter of the nanopore is 1 nm or less (0.94 nm), the amount of the blockade signal derived from dNTP is stabilized and a clear signal can be detected without being buried in noise as compared with the case of larger than 1 nm as in Comparative Example 1.

### Experimental Example 2: Change of Measurement Solution

### Example 2

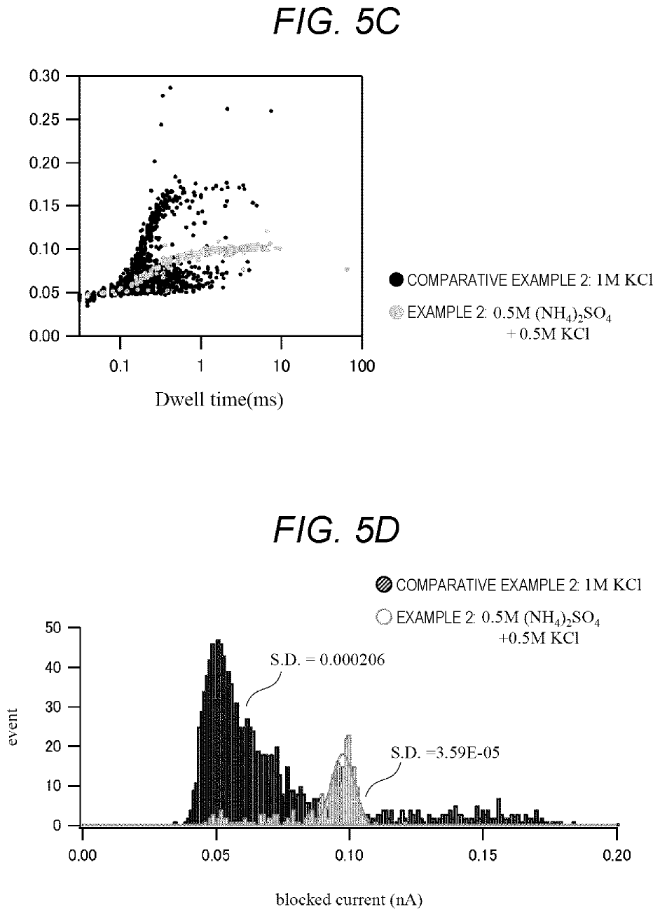

In Example 2, as the nanopore forming solution, a 0.5 M (NH₄)₂SO₄+0.5 M KCl+10 mM Tris-HCl solution (pH 7.5) was used to form a nanopore with a diameter of about 0.9 nm. Here, the time change in a current value after addition of 100 μM dCTP was measured without replacement with another solution. Thereafter, the time change of a baseline current was measured. The results are illustrated in FIG. 5B.

### Comparative Example 2

In Comparative Example 2, a nanopore with a diameter of about 0.9 nm was formed in the same manner as in Example 2, and then the nanopore forming solution was discharged and replaced with a 1 M KCl solution as the measurement solution. After the replacement with the measurement solution, the time change of a current value after addition of 100 μM dCTP was measured. The results are illustrated in FIG. 5A.

### (Results)

FIG. 5A illustrates the time change of a current value after addition of 100 μM dCTP in 1 M KCl in Comparative Example 2. As illustrated in FIG. 5A, it can be seen that the current value is in a range of about 0.05 to 0.55 nA.

FIG. 5B illustrates the time change of a current value after addition of 100 μM dCTP in a 0.5 M (NH₄)₂SO₄+0.5 KCl+10 mM Tris-HCl solution (pH 7.5) in Example 2. As illustrated in FIG. 5B, it can be seen that the current value is in a range of about 0.04 to 0.3 nA. As can be seen from FIGS. 5A and 5B, in Example 2, the range of the current value is narrow and the variation is small as compared with Comparative Example 2.

FIG. 5C is a scatter diagram of the blockade amount and blockade time of the dCTP-derived blockade signal obtained in Example 2 and Comparative Example 2. As illustrated in FIG. 5C, it was confirmed that a variation in the blocking amount of Example 2 was clearly smaller than a variation in the blockade amount of Comparative Example 2.

FIG. 5D is a histogram of the blockade amount in Example 2 and Comparative Example 2. As can be seen from FIG. 5D, in the 0.5 M (NH₄)₂SO₄+0.5 KCl+10 mM Tris-HCl solution (pH 7.5) used in Example 2, the dispersion of the blockade amount of 1 M dCTP of Comparative Example 2 is small. Further, it is found that the dispersion of the blockade amount is reduced by about 6 times the difference.

From the results of Experimental Examples 1 and 2 described above, it was confirmed that the signal derived from dNTP could be obtained by setting the nanopore diameter to 1 nm or less, even in a solution containing ammonium ions and sulfate ions. In particular, it was confirmed that the dispersion of the blockade signal derived from dNTP could be suppressed by using the measurement solution containing (NH₄)₂SO₄ at the time of ionic current measurement.

### Experimental Example 3: Comparison of Blockade Amounts of Four Nucleotides

When the base sequence of DNA is analyzed by using the ionic current, it is necessary to discriminate bases based on the blockade amounts when each of the four kinds of nucleotides constituting DNA passes through the nanopore. However, in the 1 M KCl solution which has been often used heretofore, the overlap of the distribution of blockade signals derived from various nucleotides is large. Accordingly, it is estimated that it is difficult to distinguish the bases based on only the blockade signal when one molecule passes.

Therefore, in this experimental example, the four kinds of nucleotides were respectively measured in the measurement solution, and the distribution of the amounts of the blockade signals were compared.

### Example 3

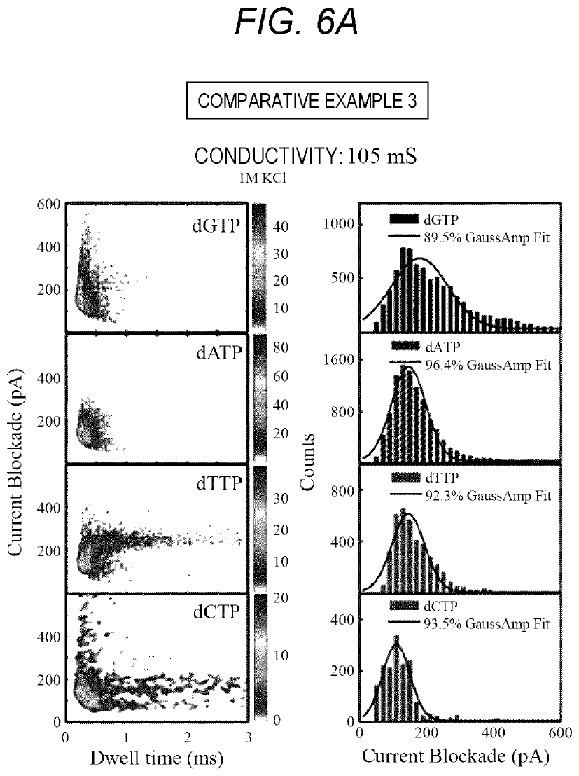

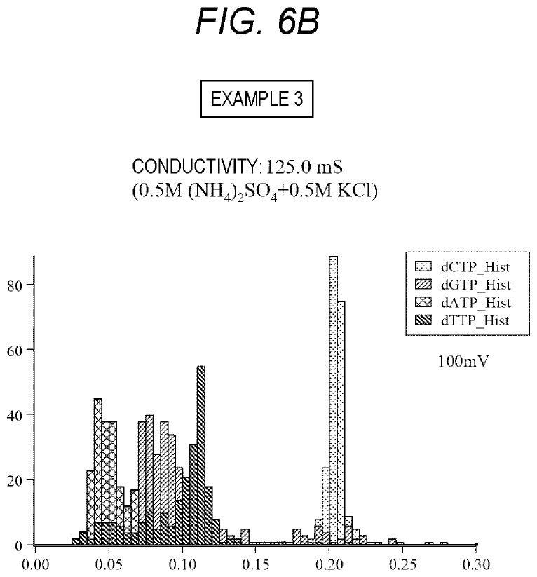

In a nanopore device prepared under the same conditions as in Example 1, a nanopore was formed. Thereafter, a 0.5 M (NH₄)₂SO₄+0.5 M KCl solution was used as the measurement solution, and solutions containing 100 μM of dCTP, dATP, dTTP, or dGTP were sequentially replaced. Then, the amounts of the blockade signals were compared. The results are illustrated in FIG. 6B.

### Comparative Example 3

Under the conditions described in NPL 2, measurement solutions containing dGTP, dATP, dTTP, or dCTP, respectively, were used to compare the amounts of the blockade signals. Specifically, the conditions of Comparative Example 3 were the same as those of Example 3 except that a nanopore was formed using a TEM device and 1 M KCl was used as the measurement solution. The results are illustrated in FIG. 6A.

### (Results)

FIG. 6A illustrates scatter diagrams and histograms of the amounts of the blockade signals derived from the nucleotides in Comparative Example 3. As illustrated in FIG. 6A, it can be seen that the values in the histograms of the blockade amounts derived from the nucleotides are superimposed on one another, and no clear separation is observed.

FIG. 6B illustrates a histogram of the blockade amount when nucleotides were measured in 0.5 M (NH₄)₂SO₄+0.5 M KCl in Example 3. As illustrated in FIG. 6B, as compared with FIG. 6A, peaks in the histogram distribution of the nucleotides are clearly different, and the overlapping of the distribution is also small. In Example 3, 0.5 M (NH₄)₂SO₄+0.5 M KCl was used as the measurement solution. Measurement at a salt concentration equal to or more than the concentration is also possible, and clearer separation can be expected.

### Modified Example

The present disclosure is not limited to the above-described embodiments, and includes various modified examples. For example, the above-described embodiments have been described in detail in order to describe the present disclosure in an easily understandable manner, and all the described configurations are not necessarily included. Furthermore, part of one embodiment can be replaced with a configuration of another embodiment. Alternatively, the configuration of another embodiment can be added to the configuration of one embodiment. Alternatively, as for part of the configuration of each of the embodiments, part of the configuration of another embodiment can be added, deleted, or displaced.

The contents of all publications and patent literatures cited herein are hereby incorporated by reference in their entirety.

### REFERENCE SIGNS LIST

1, 2 biomolecule analysis apparatus
100, 200 nanopore device
101 nanopore
102 thin film
103 electrolyte solution
104A first liquid tank
104B second liquid tank
105A first electrode
105B second electrode
106 ammeter
107 power source
108 computer
109 biomolecule
110 biopolymer degradation mechanism

1. A biomolecule analysis method comprising:
preparing a biomolecule analysis device that includes a thin film having a nanopore with a diameter in a range of ±20% of a diameter of a biomolecule, a first liquid tank and a second liquid tank separated by the thin film, a first electrode disposed in the first liquid tank, a second electrode disposed in the second liquid tank, and a biopolymer degradation mechanism that degrades a biopolymer into the biomolecule;
degrading a biopolymer into the biomolecule in the biopolymer degradation mechanism; and
applying a voltage between the first electrode and the second electrode in a state where a measurement solution is enclosed in the first liquid tank and the second liquid tank, and measuring a current flowing between the first electrode and the second electrode,
wherein the measurement solution contains ammonium ions and sulfate ions.

2. The biomolecule analysis method according to claim 1, further comprising, applying, before preparing the biomolecule analysis device, a voltage between the first electrode and the second electrode, in a state where a nanopore forming solution is enclosed in the first liquid tank and the second liquid tank, to form the nanopore in the thin film,
wherein the nanopore forming solution contains ammonium ions and sulfate ions.

3. The biomolecule analysis method according to claim 1, wherein the biopolymer is a nucleic acid, the biomolecule is a nucleotide, and the diameter of the nanopore is 1 nm or less.

4. The biomolecule analysis method according to claim 1, wherein the diameter of the nanopore is in a range of ±10% of the diameter of the biomolecule.

5. The biomolecule analysis method according to claim 1, wherein the measurement solution is an ammonium sulfate solution, and
the ammonium sulfate has a concentration of 0.01 M or more and a saturation concentration or less.

6. The biomolecule analysis method according to claim 5, wherein the ammonium sulfate has a concentration of 0.1 M or more and a saturation concentration or less.

7. The biomolecule analysis method according to claim 6, wherein the ammonium sulfate has a concentration of 1 M or more and a saturation concentration or less.

8. The biomolecule analysis method according to claim 1, wherein the measurement solution contains ammonium sulfate and another salt as salts, and
a ratio of a concentration of the ammonium sulfate to a total concentration of the salts is 5% or more and less than 100%.

9. The biomolecule analysis method according to claim 8, wherein the ratio of the concentration of the ammonium sulfate to the total concentration of the salts is 25% or more and less than 100%.

10. The biomolecule analysis method according to claim 9, wherein the ratio of the concentration of the ammonium sulfate to the total concentration of the salts is 50% or more and less than 100%.

11. The biomolecule analysis method according to claim 1, wherein the thin film contains at least one of SiN, SiO, or Si.

12. A biomolecule analyzing reagent for use in analyzing a biomolecule by allowing the biomolecule to pass through a nanopore with a diameter in a range of ±20% of a diameter of the biomolecule,
wherein the biomolecule analyzing reagent contains ammonium ions and sulfate ions.

13. The biomolecule analyzing reagent according to claim 12, wherein the biomolecule analyzing reagent is further used in degrading the biopolymer into the biomolecules.

14. The biomolecule analyzing reagent according to claim 12, wherein the biomolecule analyzing reagent is an ammonium sulfate solution, and
the ammonium sulfate has a concentration of 0.01 M or more and a saturation concentration or less.

15. The biomolecule analyzing reagent according to claim 14, wherein the ammonium sulfate has a concentration of 0.1 M or more and a saturation concentration or less.

16. The biomolecule analyzing reagent according to claim 15, wherein the ammonium sulfate has a concentration of 1 M or more and a saturation concentration or less.

17. The biomolecule analyzing reagent according to claim 12, wherein the biomolecule analyzing reagent contains ammonium sulfate and another salt as salts, and
a ratio of a concentration of the ammonium sulfate to a total concentration of the salts is 5% or more and less than 100%.

18. The biomolecule analyzing reagent according to claim 17, wherein the ratio of the concentration of the ammonium sulfate to the total concentration of the salts is 25% or more and less than 100%.

19. The biomolecule analyzing reagent according to claim 18, wherein the ratio of the concentration of the ammonium sulfate to the total concentration of the salts is 50% or more and less than 100%.

20. A biomolecule analysis device comprising:
a biopolymer degradation mechanism configured to degrade a biopolymer into a biomolecule;
a thin film in which a nanopore with a diameter in a range of ±20% of a diameter of the biomolecules is to be formed;
a first liquid tank and a second liquid tank that are separated by the thin film and contain an electrolyte solution;
a first electrode disposed in the first liquid tank; and
a second electrode disposed in the second liquid tank,
wherein the electrolyte solution contains ammonium ions and sulfate ions.
